(19) United States
(12) Patent Application Publication
(10) Pub. No.: US 2024/0142951 A1
(43) Pub. Date: May 2, 2024

(54) MATERIAL DATA PROCESSING DEVICE AND MATERIAL DATA PROCESSING METHOD

(71) Applicant: Proterial, Ltd., Tokyo (JP)

(72) Inventors: Asami OYA, Tokyo (JP); Makoto ONO, Tokyo (JP)

(21) Appl. No.: 18/385,755

(22) Filed: Oct. 31, 2023

(30) Foreign Application Priority Data
Nov. 1, 2022 (JP) 2022-175876

(51) Int. Cl. G05B 19/418

(52) U.S. Cl. CPC G05B 19/4183; G05B 19/41885

(57) ABSTRACT

A material data processing device using a computer is provided with a regression model creation processing unit that performs machine learning using, out of process data, composition data, characteristics data, and microstructure data, two or more data including the structure data, and creates a regression model representing a correlation between respective data, an estimation processing unit that estimates, by using the regression model, the process data, the composition data, the characteristics data, or the microstructure data, having been used for machine learning, wherein the microstructure data includes a feature amount based on a magnetization temperature dependence during heating, and a feature amount based on a magnetization temperature dependence during cooling; and a temperature type selection means that selects use of either one or both of the feature amount based on the magnetization temperature dependence during heating or the feature amount based on the magnetization temperature dependence during cooling as the microstructure data to be used for the machine learning. The material data processing method includes performing the machine learning, and creating the regression model, selecting the use of either one or both of the feature amount based on the magnetization temperature dependence during heating or the feature amount based on the magnetization temperature dependence during cooling as the microstructure data to be used for the machine learning.

### CROSS-REFERENCE TO RELATED APPLICATIONS

The present patent application claims the priority of Japanese patent application No. 2022-175876 filed on Nov. 1, 2022, and the entire contents thereof are hereby incorporated by reference, the entire contents of which are incorporated herein by reference.

### TECHNICAL FIELD

The present invention relates to a material data processing device and a material data processing method, more particularly, to a material data processing device using a computer and a computer-performed material data processing device.

### BACKGROUND OF THE INVENTION

In recent years, so-called “Materials Informatics”, which uses information science such as data mining to search for new and alternative materials efficiently, has attracted attention. In Japan, material development through so-called “Materials Integration” is also being studied. “Materials Integration” is defined as a comprehensive materials technology tool that aims to support materials research and development by integrating scientific technologies such as theory, experiment, analysis, simulation, and database, into materials science achievements.

Patent Literature 1 discloses the use of a learned model obtained by machine learning for the correspondence between input information including design conditions of a material to be designed and output information including material property values. Non-Patent Literature 1 also describes predicting the structure and properties of a material based on the composition of the material and the manufacturing conditions (process) of the material, from which the performance of the material is further predicted.
Citation List Patent Literature 1: WO 2020/090848
Non-Patent Literature 1: Toshihiko Koseki, “Materials data and Materials Integration System” Journal of Information Processing and Management, Vol. 59, No. 3, p. 165 (2016)

### SUMMARY OF THE INVENTION

By the way, data relating to the structure (i.e., microstructure) of materials (hereinafter referred to as “structure data”) are obtained through measurement and observation using, e.g., X-ray diffraction, optical microscopy, and scanning electron microscopy. The reliability of the structure data (i.e., microstructure data) obtained by such measurement and observation is highly dependent on the skill of the person performing the measurement and observation, and this can affect the estimation accuracy in estimation using machine learning.

Therefore, it is an object of this invention to provide a material data processing device and a material data processing method that enables easy data acquisition and high-precision estimation.

To solve the problems described above, one aspect of the invention provides a material data processing device using a computer, comprising:
a regression model creation processing unit that performs machine learning using, out of process data including information on manufacturing conditions for manufacturing individual samples, composition data including information on composition of the individual samples, characteristics data including information on characteristics of the individual samples, and microstructure data including information on microstructure of the individual samples, two or more data including the microstructure data, and creates a regression model representing a correlation between respective data;
an estimation processing unit that estimates, by using the regression model, the process data, the composition data, the characteristics data, or the microstructure data, having been used for machine learning, wherein the microstructure data includes a feature amount based on a magnetization temperature dependence during heating, and a feature amount based on a magnetization temperature dependence during cooling; and
a temperature type selection means that selects use of either one or both of the feature amount based on the magnetization temperature dependence during heating or the feature amount based on the magnetization temperature dependence during cooling as the microstructure data to be used for the machine learning.

To solve the problems described above, another aspect of the invention provides a computer-performed material data processing method, comprising:
performing machine learning using, out of process data including information on manufacturing conditions for manufacturing individual samples, composition data including information on composition of the individual samples, characteristics data including information on characteristics of the individual samples, and microstructure data including information on microstructure of the individual samples, two or more data including the microstructure data, and creating a regression model representing a correlation between respective data;
estimating, by using the regression model, the process data, the composition data, the characteristics data, or the microstructure data, having been used for machine learning, wherein the microstructure data includes a feature amount based on a magnetization temperature dependence during heating, and a feature amount based on a magnetization temperature dependence during cooling; and selecting use of either one or both of the feature amount based on the magnetization temperature dependence during heating or the feature amount based on the magnetization temperature dependence during cooling as the microstructure data to be used for the machine learning.

To solve the problems described above, still another aspect of the invention provides a material data processing device using a computer, comprising:
a regression model creation processing unit that performs machine learning using, out of process data including information on manufacturing conditions for manufacturing individual samples, composition data including information on composition of the individual samples, characteristics data including information on characteristics of the individual samples, and microstructure data including information on microstructure of the individual samples, two or more data including the microstructure data, and creates a regression model representing a correlation between respective data; and
an estimation processing unit that estimates, by using the regression model, the process data, the composition data, the characteristics data, or the microstructure data, having been used for machine learning, wherein the microstructure data includes a feature amount based on a magnetization temperature dependence during heating, and a feature amount based on a magnetization temperature dependence during cooling.

### Advantageous Effects of the Invention

According to the invention, it is possible to provide a material data process estimation device and a material data process estimation method that enables easy data acquisition and high-precision estimation.

### BRIEF DESCRIPTION OF DRAWINGS

FIG. 1 is a schematic diagram of a material data processing device according to one embodiment of the present invention.

FIG. 2 is an explanatory diagram of a thermogravimetric measurement device.

FIG. 3A is a graph showing an example of measured data obtained by the thermogravimetric measurement device.

FIG. 3B is a graph showing the measured data in FIG. 3A which is divided into measured data during heating and measured data during cooling with temperature on the horizontal axis.

FIG. 3C is a graph showing the first-order derivative of the curve shown in FIG. 3B at temperature.

FIG. 4 is a diagram showing an example of the overall database.

FIG. 5 is a diagram showing an example of a candidate value selection screen.

FIG. 6 is a diagram showing an example of a temperature type selection screen.

FIG. 7A is an explanatory diagram of a training data extraction processing.

FIG. 7B is an explanatory diagram of a regression model creation processing.

FIG. 7C is an explanatory diagram of an estimation processing.

FIG. 8 is a flowchart showing a control flow of a material data processing method according to the embodiment of the present invention.

FIG. 9 is a flowchart showing a data acquisition processing.

FIG. 10 is a flowchart showing a feature amount extraction processing.

FIG. 11 is a flowchart showing a temperature type selection processing.

FIG. 12 is a flowchart showing the training data extraction processing.

FIG. 13A is a flowchart of the regression model creation processing.

FIG. 13B is a flowchart of the estimation processing.

FIG. 14A is a diagram showing an example of a variable setting screen.

FIG. 14B is a diagram showing an example of an evaluation value display screen.

### DETAILED DESCRIPTION OF THE INVENTION

### Embodiment

An embodiment of the invention will be described below in conjunction with the appended drawings.

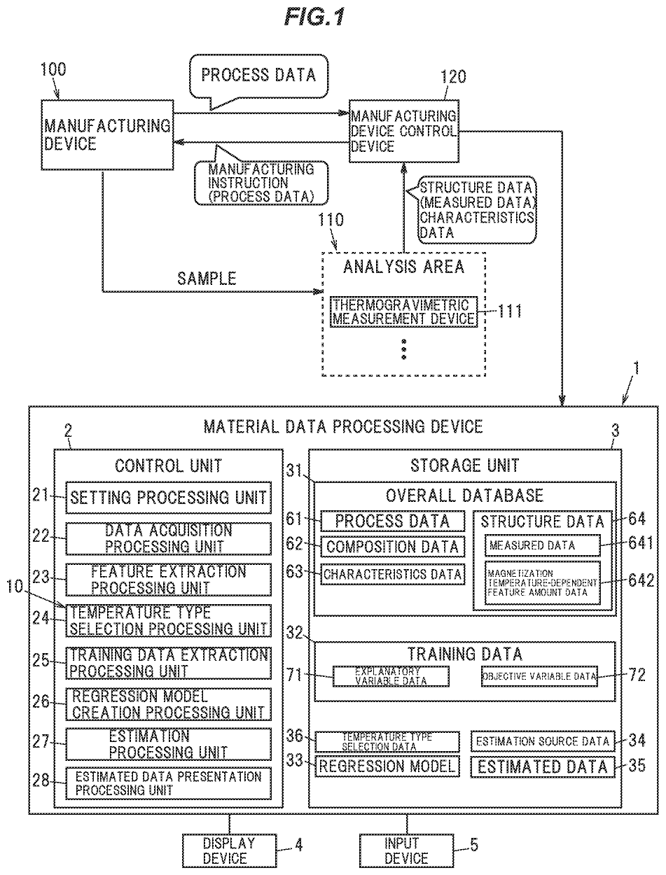

FIG. 1 is a schematic diagram of a material data processing device 1 according to one embodiment of the present invention. In addition to the material data processing device 1, FIG. 1 also shows a manufacturing device 100, an analysis area 110, and a manufacturing device control device 120.

(Target Product)

In the present embodiment, the case in which the product to be manufactured is ceramics will be explained. Furthermore, the case in which the ceramics to be manufactured are ferrite magnets, which are magnetic materials, is explained in the present embodiment. Ferrite magnets are manufactured by using metallic oxides (iron oxide) and inorganic salts of metals (strontium carbonate) as raw materials (elementary materials) and going through a mixing process, a calcining process, a fine grinding process, a molding process, and a firing process (also called sintering process). However, the products to be manufactured are not limited to magnetic materials such as ferrite magnets, rare earth magnets, or ceramic materials, but the present invention can also be applied to composite materials using resin or rubber, and the like, e.g., for a sheath of electric wires, etc. In addition, the sample to be analyzed for characteristics and structure in the present embodiment does not have to be the final product to be manufactured, but may be a semi-finished product (intermediate product).

(Explanation of Data Used for Machine Learning)

The material data processing device 1 performs machine learning using process data 61, composition data 62, characteristics data 63, and structure data 64, and estimates the desired data based on the results of the machine learning. The following is a detailed description of each of the data used for machine learning.

The process data 61 is data that includes information on the manufacturing conditions for producing individual samples. For example, the process data 61 includes set values and actual measured values for temperature, processing time, motor rotation speed, etc. in the manufacturing device 100. In the case of manufacturing magnetic materials such as ferrite magnets, the process data 61 preferably includes at least parameters that define the heat treatment conditions.

The composition data 62 includes information on the types of elements contained in individual samples and the composition ratios of the elements, for example, the content amount (composition ratio) of the materials used.

The characteristics data 63 is data that includes information on the characteristics of individual samples. When producing magnetic materials such as ferrite magnets, the characteristics data 63 preferably includes information on at least one of the following: residual flux density Bᵣ, coercive force Ha, saturation magnetization, and magnetic permeability.

The structure data 64 is then data that includes information on the structure of individual samples. When magnetic materials such as ferrite magnets are produced, the structure data 64 preferably includes parameters that define the crystal structure of the main phase. In addition, the structure data 64 includes information on a feature amount (Curie temperature T_{C}, etc.) based on the magnetization temperature dependence during heating and cooling. The details of this point are described below.

(Manufacturing Device 100 and Manufacturing Device Control Device 120)

The manufacturing device 100 is, for example, a device for manufacturing ferrite magnets. The manufacturing device control device 120 is a device that gives manufacturing instructions and various settings to the manufacturing device 100, monitors the production status of the manufacturing device 100, and collects various data during production, etc. The manufacturing device control device 120 is composed of a personal computer, for example.

The manufacturing device control device 120 receives the process data 61 from the manufacturing device 100 and receives the structure data 64 including the characteristics data 63 and measured data 641 by the thermogravimetric measurement device 111 from the analysis area 110, which will be described later. The data may be exchanged between the manufacturing device 100 or an analysis area 110 and the manufacturing device control device 120 using a USB memory or other storage medium. If the manufacturing device control device 120 has process information or the like as setting information, manufacturing instruction information, etc., it may be configured to acquire the information in its possession as the process data 61. For the composition data 62, the data inputted on the side of the manufacturing device 100 may be received by the manufacturing device control device 120, the information of composition data 62 may be inputted by the manufacturing device control device 120, or the information of composition data 62 may be directly inputted by the material data processing device 1. The manufacturing device control device 120 transmits each data received to the material data processing device 1. The details of each data are described below.

The manufacturing device control device 120 is configured to be able to output manufacturing instructions to the manufacturing device 100, and to reflect the estimated process data 61 (estimated data 35 to be described below) in the process of the manufacturing device 100 when the process is estimated by the material data processing device 1. In this embodiment, various data are transmitted to the material data processing device 1 via the manufacturing device control device 120, but the data may be configured to be output directly from the manufacturing device 100 or the analysis area 110 to the material data processing device 1. In addition, a management device for managing each data to be used for machine learning may be provided separately from the manufacturing device control device 120, and each data may be configured to be transmitted to the material data processing device 1 from the management device.

(Analysis Area 110 and Structure Data 24)

The analysis area 110 is an area where the structure and characteristics of individual samples produced by the manufacturing device 100 are analyzed. In the analysis area 110, the analysis of the structure and characteristics is performed using various devices for analyzing the structure and characteristics of individual samples. The “area” of the analysis area 110 does not represent a specific location, but rather a conceptual area that groups analytical devices and other devices. In other words, respective devices for analysis need not be located together in one place.

The details of the structure data 64 are explained here. The structure data can include information on the proportion of each phase comprising the material, crystal structure, molecular structure, single crystalline/polycrystalline/amorphous distinction, grain shape and size in the polycrystalline case, crystal orientation, grain boundaries, twinning or stacking faults, type and density of defects such as transitions, segregation of solute elements at grain boundaries and within grains, etc. In the present embodiment, information on “magnetic phase transitions,” which in the past has generally been treated as information that defines the “properties” of a material (i.e., the characteristics data 63), is used as the structure data 64. In other words, the structure data 64 includes feature amount based on the magnetization temperature dependence in individual materials.

Next, the “feature amount based on magnetization temperature dependence” will be explained. A typical example of a magnetic phase transition is the “ferromagnetic-paramagnetic transition”. The temperature at which such a magnetic phase transition occurs is called the Curie temperature (T_{C}) or Curie point. The Curie temperature of a material strongly depends on the crystal structure and composition of the phases that make up the material, etc., and can be used as the structure data 64. The acquisition of “feature amount based on magnetization temperature dependence” has the advantage that the quality of data is unlikely to fluctuate depending on the personal skills of the person collecting the data, and the data can be acquired mechanically. The “feature amount related to the magnetic phase transition” is a feature amount that indicates the structural characteristics caused by the “ferromagnetic-paramagnetic transition” and can be defined by the Curie temperature. Here, “ferromagnetism” in a broad meaning means “strong magnetism” which shall include not only “ferromagnetism” in a narrow meaning but also “ferrimagnetism”. In addition, a feature amount indicating the structural characteristics caused by the “antiferromagnetic-paramagnetic transition” may also be used. Such a feature amount is defined by Neel temperature. In other words, examples of feature amounts based on magnetization temperature dependence include feature amounts related to the magnetic phase transition, more specifically, at least one of the Curie temperature and the Neel temperature.

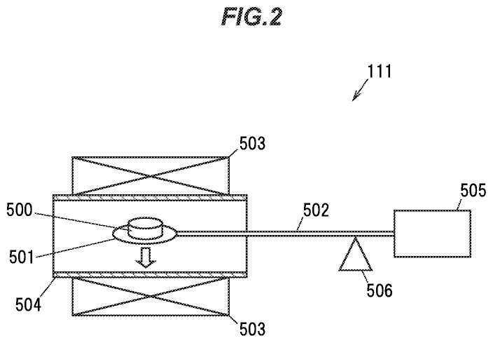

The Curie temperature can be measured using a thermogravimetric measurement device (TG: Thermogravimetry) 111, which is simple and sensitive. As shown in FIG. 2, the thermogravimetric measurement device 111 has a beam section 502 with a holder 501 at one end that holds a sample (measurement sample) 500 in a container, an electric furnace 504 with a heater 503 that heats the sample 500, and a gravimetric section 505 connected to the other end of the beam section 502 to detect weight changes of the sample 500. The beam section 502 is supported by a support 506 that serves as a fulcrum.

In the thermogravimetric measurement device 111, the gravimetric section 505 measures the weight changes associated with reactions such as pyrolysis that occur in the sample 500 when the sample 500 is heated. When extracting features related to magnetic phase transition, a magnetic field gradient is applied externally to the sample 500 during measurement. This can exert a magnetic attractive force on the sample 500 as indicated by the white arrow in FIG. 2. As a result, the magnetic attractive force is superimposed on the weight of the sample 500, and the “weight” value measured by the gravimetric section 505 will include the magnetic attractive force exerted on the sample 500. The magnetic attractive force corresponds to the magnitude of the “magnetization” of the sample 500. Therefore, when a phase transition from ferromagnetism to paramagnetism occurs in the sample 500, the magnetization of the sample 500 changes rapidly, and the phase transition can be detected as a change in “weight” measured by the gravimetric section 505. Hereafter, the measurement value measured by the gravimetric section 505 is referred to as the TG measured value w.

In the example shown in FIG. 2, the sample 500 and the gravimetric section 505 are arranged horizontally, but may be arranged vertically. The thermogravimetric measurement device 111 may also have an additional function that allows differential thermal analysis (DTA) or differential scanning calorimetry (DSC) to be performed simultaneously. In this case, the sample 500 and a reference sample may be set in the device for measurement, and a paramagnetic material such as alumina (a material that does not develop ferromagnetism over the entire measurement temperature range) is preferably used as the reference sample.

The configuration for applying a magnetic field gradient to the sample 500 can be of any configuration as long as reproducibility between measurements of individual samples is ensured, and can be easily achieved by installing a permanent magnet such as a rare earth magnet in the device, for example. The size of the magnetic field gradient may be appropriately selected according to the amount of the sample 500, e.g., 0.1 mT/mm. Since phase transitions can be detected with higher sensitivity when the magnetic field gradient is larger, a magnetic field gradient of 0.5 mT/mm or more is preferred, and a magnetic field gradient of 1 mT/mm or more is even more preferred.

The sample 500 is placed in a container (pan) made of alumina, for example, and set in holder 501. If measurement materials with magnetic anisotropy, such as Nd—Fe—B sintered magnets are measured in bulk form, the magnetic attractive force may fluctuate depending on the direction in which they are set. To suppress such fluctuations, the sample 500 in pulverized powder form may be used. In this case, the pulverized particle size may be selected according to the material to be measured, e.g., 500 μm or less. When easily oxidizable materials are measured, the pulverized particle size may be coarser to suppress weight gain due to oxidation of the sample 500 caused by a small amount of oxygen contained in the inert gas during the measurement. In the case of easily oxidizable materials such as rare earth magnets, for example, an inert gas such as argon gas may be employed as the atmosphere during measurement to avoid weight changes due to oxidation reactions during measurement and the generation of a new ferromagnetic phase due to reactions. A getter material or the like to remove impurities in the inert gas may also be incorporated in the device.

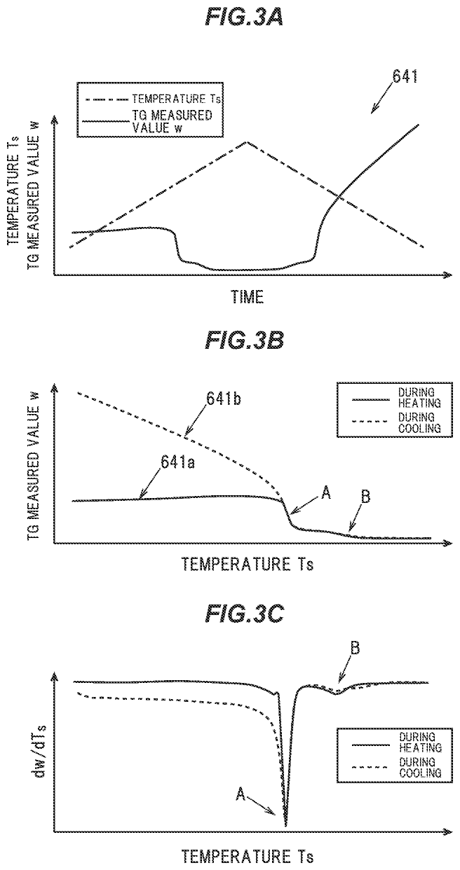

In the measurement by the thermogravimetric measurement device 111, the temperature Ts and the TG measured value w of the sample installation section are measured while gradually heating from room temperature to a predetermined temperature and then gradually cooling to room temperature. An example of the measured data 641 obtained by the thermogravimetric measurement device 111 is shown in FIG. 3A. In FIG. 3A, the measured data 641 obtained from the thermogravimetric measurement device 111 is shown as a solid line and the temperature profile is shown as a dashed line. The TG measured value w is the superimposed value of the weight of the sample 500, the weight of the alumina container (pan), and the magnetic attraction. Since the weight of the pan and the sample 500 does not change with temperature, the change in the TG measurement w corresponds to a change in the magnitude of the magnetic force received by the sample 500, i.e., a change in the magnitude of the magnetization of the sample 500.

FIG. 3B is a graph showing the temperature dependence of the TG measured value w, based on the measured data 641 obtained by the thermogravimetric measurement device 111. In the graph in FIG. 3B, the TG measured value w changes abruptly at a given temperature (arrows A and B in the drawing). This abrupt change in the TG measurement w is due to the ferromagnetic-paramagnetic transition of the phase (ferromagnetic phase) contained in the sample. The amount of change in the TG measurement w reflects the magnetization and volume ratio of the ferromagnetic phase in the sample.

FIG. 3C is a graph showing values in the graph of FIG. 3B differentiated by temperature Tₛ. The temperature that takes a minimum value in FIG. 3C (arrows A and B in the drawing) is the Curie temperature T_{C}. The method of determining the Curie temperature T_{C} is not limited to this, and the Curie temperature T_{C} may be determined by other methods. However, since the Curie temperature T_{C} is used for machine learning as the structure data 64 in this embodiment, the method for obtaining the Curie temperature T_{C} is preferably unified.

From FIGS. 3B and 3C, it can be seen that the Curie temperature T_{C} is different during heating and cooling. Hereafter, the Curie temperature T_{C} during heating is referred to as the Curie temperature T_{C_H} during heating, and the Curie temperature during cooling T_{C} is referred to as the Curie temperature during cooling T_{C_C}. The Curie temperature during heating T_{C_H} is the transition temperature at which a ferromagnetic material changes to a paramagnetic material when heated from room temperature to a predetermined temperature. Meanwhile, the Curie temperature during cooling T_{C_C} is the transition temperature at which a ferromagnetic material changes from a paramagnetic material to a ferromagnetic material when it is cooled from a heated state to a room temperature to a predetermined temperature.

In one measured data 641 (i.e., one sample), there may be more than one Curie temperature during heating T_{C_H} and more than one Curie temperature during cooling T_{C_C}, respectively. In the case of a ferrite magnet, there are two or more Curie temperatures for heating T_{C_H} and two or more Curie temperatures for cooling T_{C_C}, respectively. In the machine learning described below, each of the multiple Curie temperatures during heating T_{C_H} and multiple Curie temperatures during cooling T_{C_C} will be treated as a single variable.

The analysis area 110 may be equipped with, for example, an X-ray diffractometer, optical microscope, etc., in addition to the thermogravimetry measurement device 111 as device for analyzing the structure of individual samples. The X-ray diffractometer is used, for example, to determine the types and proportions of phases (compounds) present in the material, lattice constants, and the like. Optical microscopy is used, for example, to measure the size of each phase. A scanning electron microscope (SEM: Scanning Electron Microscope) may also be used instead of an optical microscope, for example. The composition of each phase may be determined, for example, by an energy dispersive X-ray spectroscopy (EDX: Energy Dispersive X-ray spectroscopy) or an electron probe micro analyzer (EPMA: Electron Probe Micro Analyzer) attached to the SEM.

(Material Data Processing Device 1)

Returning to FIG. 1, the material data processing device 1 has at least a control unit 2, a storage unit 3, and a communication unit (not shown). The control unit 2 comprehensively controls the entire material data processing device 1, and the storage unit 3 stores information, etc. necessary for the various processes described below by the control unit 2. The material data processing device 1 is, for example, a computer such as a server device, and is equipped with an arithmetic element such as a CPU, memory such as RAM and ROM, storage such as a hard disk, and a communication interface that is a communication device such as a LAN card.

The control unit 2 has a setting processing unit 21, a data acquisition processing unit 22, a feature amount extraction processing unit 23, a temperature type selection processing unit 24, a training data extraction processing unit 25, a regression model creation processing unit 26, an estimation processing unit 27, and an estimated data presentation processing unit 28. Details of each part are described below.

The storage unit 3 is realized by a predetermined storage area of a memory or storage device. The material data processing device 1 also has a display device 4 and an input device 5. The display device 4 is, for example, a liquid crystal display, and the input device 5 is, for example, a keyboard and a mouse. The display device 4 may be configured as a touch panel, and the display device 4 may also serve as the input device 5. The display device 4 and input device 5 may be configured separately from the material data processing device 1 and be capable of communicating with the material data processing device 1 by wireless communication or the like. In this case, the display device 4 or input device 5 may comprise a portable terminal such as a tablet or smartphone.

(Setting Processing Unit 21)

The setting processing unit 21 performs setting processing for various settings of the material data processing device 1. The setting processing unit 21 can, for example, set the method of data acquisition by the data acquisition processing unit 22, the date and time of data acquisition, and other information relating to various controls. In addition, the setting processing unit 21 can register, update, delete, etc., various information to be stored in the storage unit 3. The input device 5 or the like can be used to input various information, etc.

(Data Acquisition Processing Unit 22)

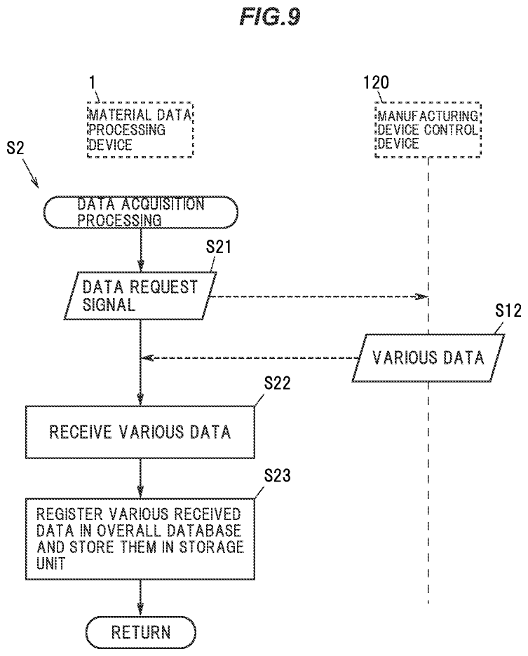

The data acquisition processing unit 22 performs data acquisition processing (see FIG. 9) to acquire each data from the manufacturing device control device 120. The data acquisition processing unit 22 performs data acquisition processing when it receives a data update signal from the manufacturing device control device 120. In the data acquisition processing, the data acquisition processing unit 22 sends a data request signal to the manufacturing device control device 120, receives each data transmitted from the manufacturing device control device 120 accordingly, and stores the data in the storage unit 3 as the overall database 31. The data acquisition processing unit 22 may also acquire data by input from the input device 5. For example, the composition data 62 may be acquired by input from the input device 5.

(Overall Database 31)

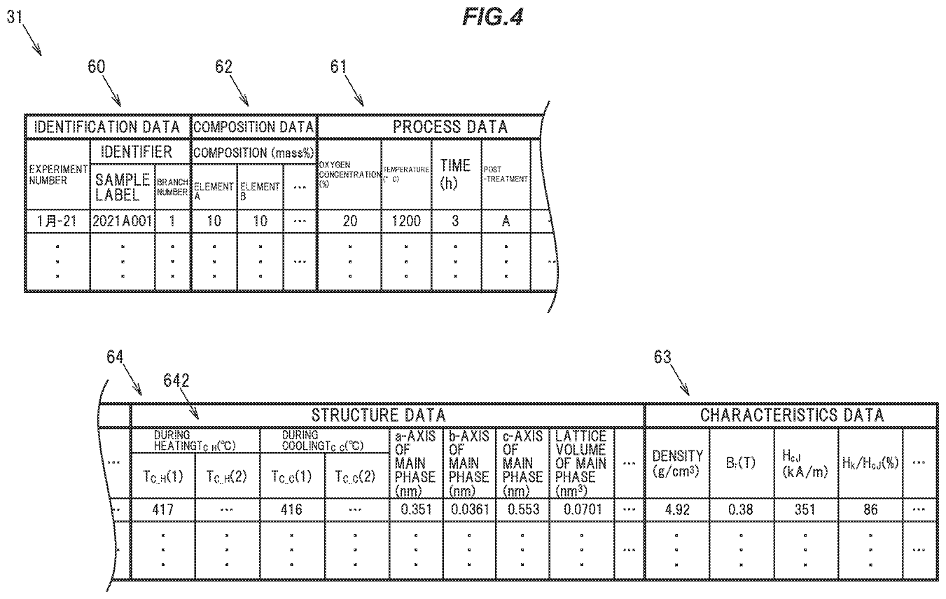

The overall database 31 is explained here. FIG. 4 is a diagram showing an example of the overall database 31. Note that FIG. 4 shows the concept of the overall database 31 and does not describe the actual experimental data.

As shown in FIG. 3, the overall database 31 is a database that collectively manages the collected data, and includes each of the data obtained from the manufacturing device control device 120 and the input device 5, namely, the process data 61, the composition data 62, the characteristics data 63, and the structure data 64. In the example shown in the drawing, in addition to these data, an identification data 60, which includes information on experiment numbers and identifiers, is also included in the overall database 31. The structure data 64 includes a magnetization temperature-dependent feature amount data 642, which is the data of the feature amount based on the magnetization temperature dependence during heating (Curie temperature during heating T_{C_H}) and the feature amount based on the magnetization temperature dependence during cooling (Curie temperature during cooling T_{C_C}). The overall database 31 may include data other than the data shown in the drawing as needed.

(Feature Amount Extraction Processing Unit 23)

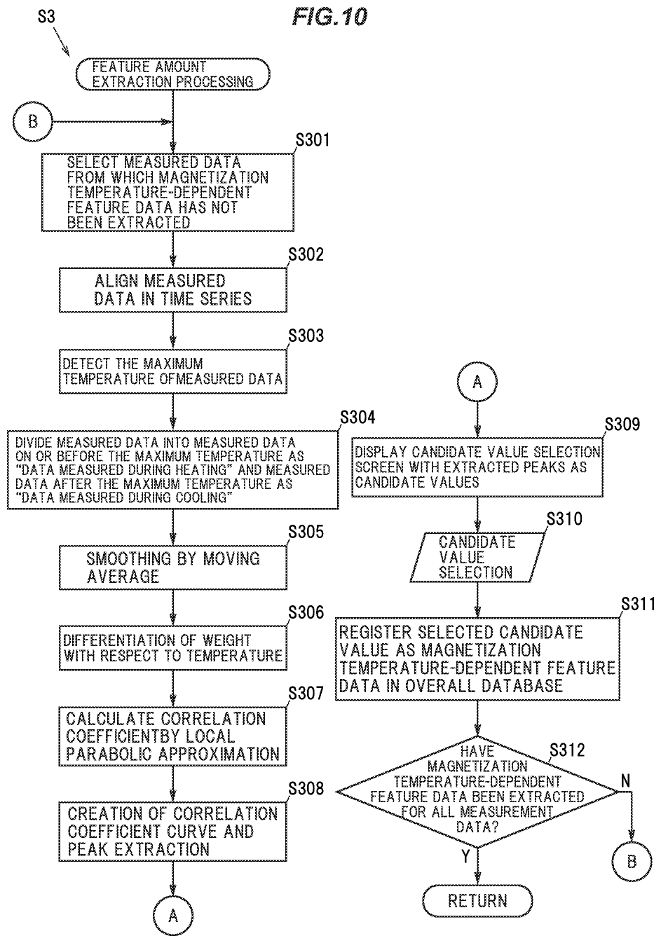

The feature amount extraction processing unit 23 performs a feature amount extraction processing (see FIG. 10) based on the measured data 641 of individual samples to extract the feature amounts based on magnetization temperature dependence during heating (Curie temperature during heating T_{C_H}) and feature amounts based on magnetization temperature dependence during cooling (Curie temperature during cooling T_{C_C}).

In the feature amount extraction process, the measured data 641 (see FIG. 3A) is divided into measured data during heating 641a and measured data during cooling 641b. In this case, it is preferable to detect the time of the highest temperature after arranging the measured data 641 in chronological order, and divide the data before the detected time of the highest temperature as the measured data during heating 641a and the data after the detected time of the highest temperature as the measured data during cooling 641b (see FIG. 3B).

The feature amount extraction processing unit 23 then extracts a feature amount based on the magnetization temperature dependence during heating (Curie temperature during heating T_{C_H}) from the measured data during heating 641a and a feature amount based on the magnetization temperature dependence during cooling (Curie temperature during cooling T_{C_C}) from the measured data during cooling 641b. More specifically, after performing data processing for noise reduction such as smoothing of each of the measured data during heating 641a and the measure data during cooling 641b, the weight derivative with respect to temperature is performed on each of the measured data 641a and 641b (see FIG. 3C).

Then, by performing peak extraction from the obtained derivative data, candidate values for the Curie temperature during heating T_{C_H} and the Curie temperature during cooling T_{C_C} are extracted. The peak extraction method includes, for example, calculating the correlation coefficient by local parabolic approximation and extracting peaks based on the obtained correlation coefficient curve.

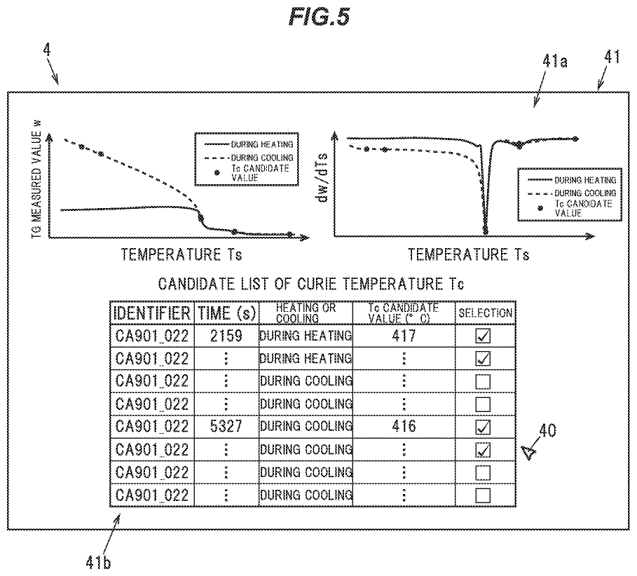

Since the measured data 641 measured by the thermogravimetric measurement device 111 contains a lot of noise, the candidate values of Curie temperature during heating T_{C_H}, and Curie temperature during cooling T_{C_C} obtained by peak extraction may contain incorrect values due to noise effects. Therefore, in the present embodiment, the user of the material data processing device 1 manually determines the values of Curie temperature during heating T_{C_H} and Curie temperature during cooling T_{C_C}. In this case, for example, the feature amount extraction processing unit 23 may be configured to support the user's selection by displaying a candidate value selection screen 41 as shown in FIG. 5 on the display device 4.

In the example of FIG. 5, the upper part of the candidate value selection screen 41 has a graph display area 41a showing the measured data 641 and a graph differentiating the measured data 641, and candidate values of Curie temperature T_{C} (Curie temperature during heating T_{C_H}, and Curie temperature during cooling T_{C_C}) are displayed in each graph. In the lower part of the candidate value selection screen 41, a list of candidate values of Curie temperature during heating T_{C_H} and Curie temperature during cooling T_{C_C} is displayed, and a selection area 41b is provided for selecting Curie temperature during heating T_{C_H} and Curie temperature during cooling T_{C_C} from the candidate values. In the illustrated example, the user moves a cursor 40 on the candidate value selection screen 41 using the input device 5 such as a mouse, and checks the checkboxes in the selection area to select the Curie temperatures during heating T_{C_H} and the Curie temperatures during cooling T_{C_C} to be used. By using the candidate value selection screen 41 as shown in FIG. 5, the user can select the Curie temperature during heating T_{C_H} and the Curie temperature during cooling T_{C_C} while viewing the graph, which makes it easier to select the Curie temperature during heating T_{C_H} and the Curie temperature during cooling T_{C_C}. The selected Curie temperatures during heating T_{C_H} and the selected Curie temperatures during cooling T_{C_C} are registered in the overall database 31 and stored in the storage unit 3 as the magnetization temperature-dependent feature amount data 642.

In this case, the Curie temperature T_{C} (Curie temperature during heating T_{C_H}, and Curie temperature during cooling T_{C_C}) was selected manually, but not only this, if the measured data 641 can be obtained with sufficiently low noise effect, the selection of candidate values can be omitted and t the Curie temperature T_{C} (Curie temperature during heating T_{C_H}, and Curie temperature during cooling T_{C_C}) may be selected.

In this embodiment, the feature amount extraction based on magnetization temperature dependence during heating and cooling was performed at the material data processing device 1. However, the invention is not limited to this, for example, the feature amount extraction based on magnetization temperature dependence during heating and cooling may be performed at the analysis area 110 or the manufacturing device control device 120.

(Temperature Type Selection Processing Unit 24)

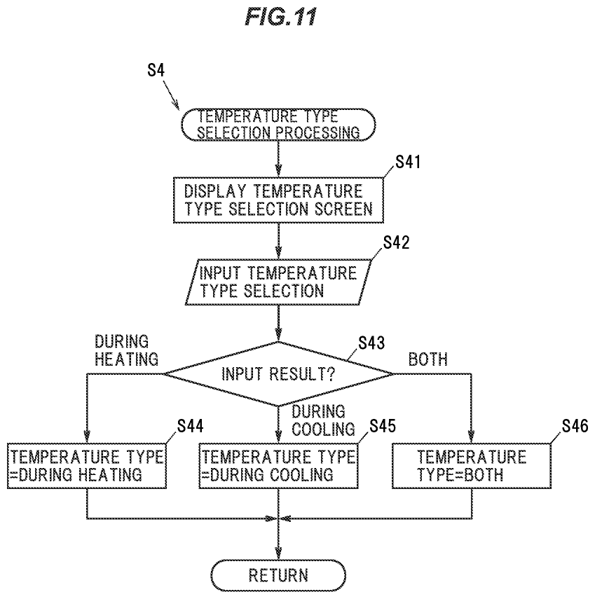

The temperature type selection processing unit 24 performs temperature type selection processing to select to use one or both of the feature amount based on the magnetization temperature dependence during heating (Curie temperature during heating T_{C_H}) or the feature amount based on the magnetization temperature dependence during cooling (Curie temperature during cooling T_{C_C}) as the structure data 64 used for machine learning (See FIG. 11).

The inventors have found that for some types of magnets, the composition may change due to oxidation or a high-temperature phase appears during heating, and that it is desirable to use a feature amount based on the magnetization temperature dependence during heating (Curie temperature during heating T_{C_H}) for these types of magnets. For those that are not affected by the appearance of high-temperature phases, etc., it is recommended to use a feature amount based on the magnetization temperature dependence during both heating and cooling, or during cooling. In the case of ferrite magnets, it was found that the estimation accuracy was most improved by using only the feature amounts based on the magnetization temperature dependence during cooling. In this way, depending on the type of magnet, etc., the estimation accuracy can be improved by selecting the feature amounts based on the magnetization temperature dependence during heating or cooling, or both, for use in machine learning.

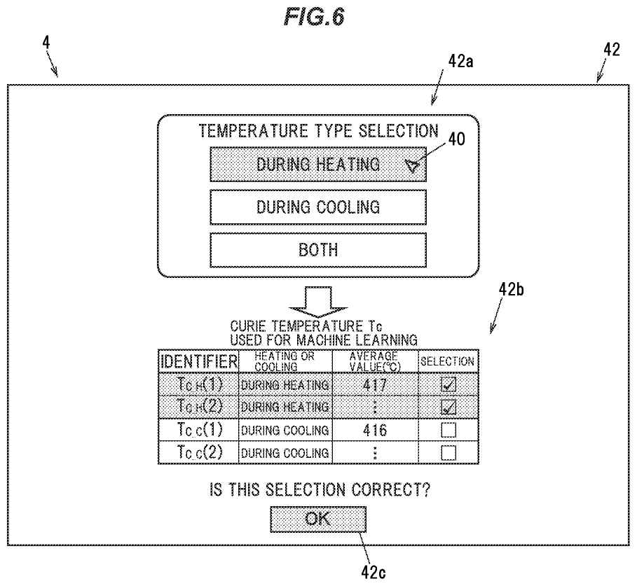

The temperature type selection processing unit 24 displays a temperature type selection screen 42 as shown in FIG. 6 on the display device 4. The temperature type selection screen 42 has a temperature type selection area 42a at the top that allows selection of heating, cooling, or both, and a selection result display area 42b at the bottom that displays the feature amount based on the magnetization temperature dependency selected in the temperature type selection area 42a. The user selects a temperature type in the temperature type selection area 42a by operating the cursor 40 with the input device 5 such as a mouse. Then, the temperature type selection processing unit 24 displays the selection result in the selection result display area 42b. By unchecking the checkboxes in the selection result display area 42b, it is possible, for example, to select only one of the Curie temperatures during heating T_{C_H}. By clicking an OK button 42c at the bottom of the screen, the selection is confirmed. The selection result is stored in the storage unit 3 as the temperature type selection data 36. The temperature type selection processing unit 24, the display device 4, the input device 5, and an user interface for input correspond to the temperature type selection means 10 of the present invention. The temperature type selection screen 42 in FIG. 6 is only an example and can be changed as needed.

(Training Data Extraction Processing Unit 25)

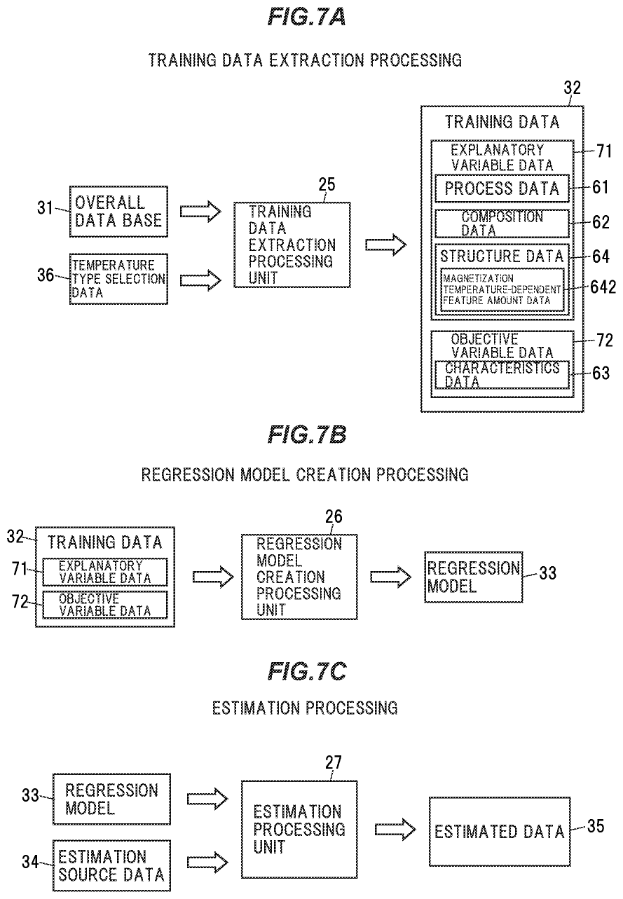

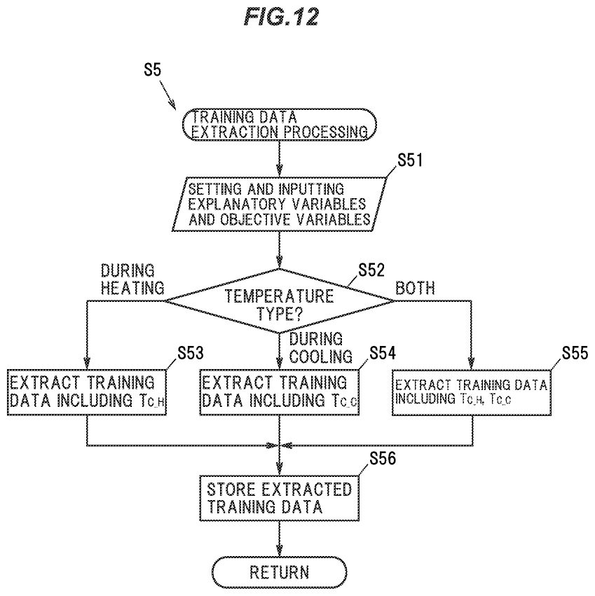

The training data extraction processing unit 25 extracts from the overall database 31 only the data to be used for machine learning as the training data 32 (see FIG. 12). As shown in FIG. 7A, in the training data extraction processing, explanatory variable data 71 (explanatory variables) and objective variable data 72 (objective variables) are extracted from the data contained in the overall database 31.

In machine learning, estimation accuracy depends on the combination of explanatory variable data 71 and objective variable data 72. In the present embodiment, as a combination that tends to increase estimation accuracy based on experience, the characteristics data 63 is used as the objective variable data 72, and data other than the characteristics data 63 (i.e., the process data 61, the composition data 62, and the structure data 64) is used as the explanatory variable data 71. It is essential to use the structure data 64 as the explanatory variable data 71 or the objective variable data 72 in the present embodiment. However, data other than structure data 64, i.e., the process data 61, the composition data 62, and the characteristics data 63, are not essential in the present embodiment and can be used as necessary.

The data extraction processing unit 25 extracts only those selected by the temperature type selection data 36 (e.g., only the feature amount based on the magnetization temperature dependence during heating (Curie temperature during heating T_{C_H})) from the structure data 64 in the overall database 31 to be the explanatory variable data 71. The extracted training data 32 is stored in the storage unit 3.

In order to avoid a decrease in estimation accuracy due to over-training, etc., the user may wish to change the selection of parameters to be used as the explanatory variable data 71 and the objective variable data 72, and repeat the creation of the regression model 33. In this case, a parameter selection screen that allows selection of parameters to be used as the explanatory variable data 71 and the objective variable data 72 may be displayed on the display device 4 to allow the user to select each parameter. The parameter selection screen is described below.

(Regression Model Creation Processing Unit 26)

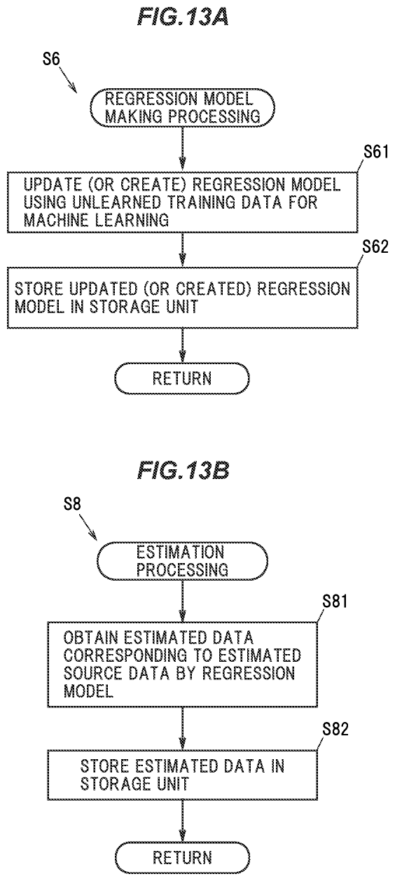

As shown in FIG. 7B, the regression model creation processing unit 26 performs machine learning using the extracted training data 32 to create a regression model 33 that shows the correlation between each parameter of the explanatory variable data 71 and each parameter of the objective variable data 72 (see FIG. 13A). In the present embodiment, the regression model creation processing unit 26 performs machine learning using two or more of the process data 61, the composition data 62, the characteristics data 63, and the structure data 64, including the structure data 64, to create the regression model 33 that shows the correlation between each data. In other words, in the present embodiment, it is essential to use the structure data 64 for machine learning.

The regression model creation processing unit 26 includes software such as a learning algorithm for learning the correlation of the parameters of the objective variable data 72 to each parameter of the input explanatory variable data 71 by itself through machine learning. The learning algorithm is not particularly limited, and any known learning algorithm can be used, such as so-called deep learning using a neural network with three or more layers. What the regression model creation processing unit 26 learns corresponds to a model structure that represents the correlation between the explanatory variable data 71 and the objective variable data 72.

More specifically, the regression model creation processing unit 26 iteratively executes learning based on a data set containing the explanatory variable data 71 and the objective variable data 72, based on the input training data 32, and automatically interprets the correlation between the explanatory variable data 71 and the objective variable data 72. Although the correlation is unknown at the start of learning, the correlation of the objective variable data 72 with respect to the explanatory variable data 71 is gradually interpreted as the learning proceeds, and the resulting learned model, i.e., the regression model 33, is used to allow for the interpretation of the correlation between the explanatory variable data 71 and the objective variable data 72.

The regression model creation processing unit 26 stores the created regression model 33 in the storage unit 3. In the present embodiment, the regression model creation processing unit 26 updates the regression model 33 every time the overall database 31 is updated. However, the invention is not limited to this, for example, the data updates may be learned together and the regression model 33 may be updated when the estimation process described below is executed. Also, if the parameters used as the explanatory variable data 71 or the objective variable data 72 are changed, the regression model 33 will be created again.

(Estimation Processing Unit 27)

The estimation processing unit 27 uses the regression model 33 created by the regression model creation processing unit 26 to perform the estimation processing to estimate any of the data used for machine learning, namely, the process data 61, the composition data 62, the characteristics data 63, or the structure data 64 (see FIG. 13B). As shown in FIG. 7C, the estimation processing unit 27 obtains an estimated data 35 based on the regression model 33 created by the regression model creation processing unit 26 and an estimation source data 34, which is the data from which the estimation is made. The obtained estimated data 35 is stored in the storage unit 3. For example, when estimating the process data 61, the estimated data 35 is the process data 61, and the estimation source data 34 is data other than the process data 61 used for machine learning. The estimation source data 34 is, for example, input by the input device 5.

(Estimated Data Presentation Processing Unit 28)

The estimated data presentation processing unit 28 performs estimated data presentation processing to present the estimated data 35. In the estimated data presentation processing, for example, the estimated data 35 are displayed on the display device 4. The estimated data presentation processing may also be configured to present data other than the estimated data 35, such as items used as the explanatory variable data 71 and the objective variable data 72 together with the estimated data 35.

(Material Data Processing Method)

### (Main Routine)

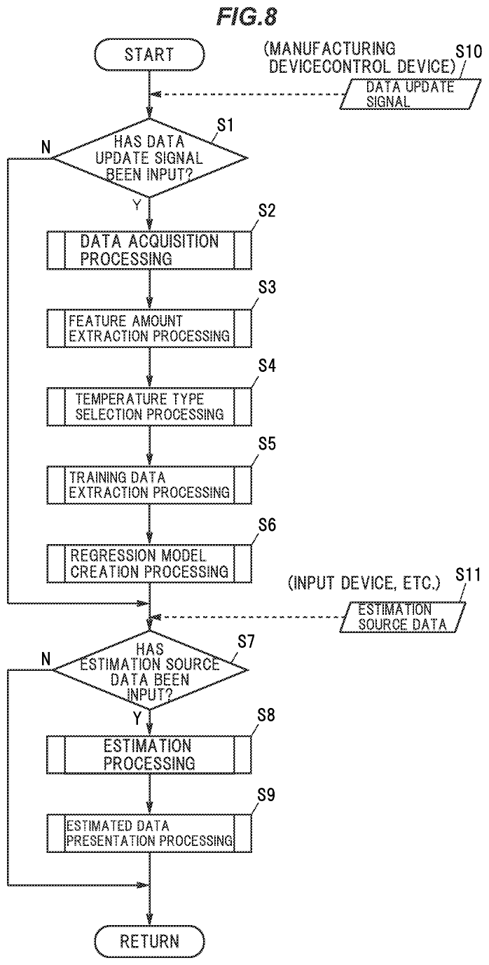

FIG. 8 is a flowchart of the material data processing method. In FIG. 8, the solid arrows represent the flow of control, and the dashed arrows represent the input and output of signals and data.

As shown in FIG. 8, when the manufacturing device control device 120 receives data from the manufacturing device 100 or the analysis area 110, it sends a data update signal to the material data processing device 1 (step S10). The control unit 2 of the material data processing device 1 determines in step S1 whether the data update signal has been input. If it is determined to be NO (N) in step S1, it proceeds to step S7. If YES (Y) is determined in step S1, it proceeds to step S2 for data acquisition processing.

In the data acquisition process of step S2, as shown in FIG. 9, the data acquisition processing unit 22 sends a data request signal to the manufacturing device control device 120 in step S21. The manufacturing device control device 120 that receives the data request signal transmits to the data acquisition processing unit 22 various data received from the manufacturing device 100 and the analysis area 110 (step S12). Thereafter, in step S22, the data acquisition processing unit 22 receives the various data. Although omitted from the drawing, the data acquisition processing unit 22 may also receive data input from the input device 5. Then, in step S23, the data acquisition processing unit 22 registers the various data received into the overall database 31 and stores it in the storage unit 3. It then returns and proceeds to step S3 in FIG. 8.

In the feature amount extraction processing of step S3, as shown in FIG. 10, in step S301, the feature amount extraction processing unit 23 refers to the overall database 31, selects the measured data 641 from which the magnetization temperature dependence data 642 has not been extracted, and in step S302, arranges the measured data 641 in time series (see FIG. 3A), and detects the highest temperature in the measured data 641 in step S303. Then, in step S304, the feature amount extraction processing unit 23 divides the measured data 641 into two parts, with the data before the time of the maximum temperature as the measured data during heating 641a and the data after the time of the maximum temperature as the measured data during cooling 641b (see FIG. 3B). Then, in step S305, the feature amount extraction processing unit 23 performs smoothing processing by moving average on the measured data during heating and cooling, and then in step S306, the derivative of weight with respect to temperature is taken (see FIG. 3C). Then, in step S307, the feature amount extraction processing unit 23 calculates a correlation coefficient by local parabolic approximation for the graph of derivatives, and in step S308, a correlation coefficient curve is created based on the calculated correlation coefficient, and peak extraction is performed based on the correlation coefficient curve.

Then, in step S309, the feature amount extraction processing unit 23 displays the candidate value selection screen 41 on the display device 4 as the candidate value of the Curie temperature T_{C} for the extracted peak (see FIG. 5). At this time, the candidate value extracted from the measured data during heating becomes the candidate value for the Curie temperature during heating T_{C_H}, and the candidate value extracted from the measured data during cooling is displayed as the candidate value for the Curie temperature during cooling T_{C_C}. Thereafter, in step S310, inputs are accepted on the candidate value selection screen 41, and in step 311, the candidate values selected in step S310 are registered in the overall database 31 as magnetization temperature-dependent feature amount data 642. Then, in step S312, it is determined whether magnetization temperature-dependent feature amount data 642 has been extracted for all measured data (whether there is data for which magnetization temperature-dependent feature amount data 642 in the overall database 31 is blank and the measured data 641 exists). If NO (N) is determined in step S312, the process returns to step S301. If YES (Y) is determined in step S312, the process returns and proceeds to step S4 in FIG. 8.

In the temperature type selection processing of step S4, as shown in FIG. 11, in step S41, the temperature type selection processing unit 24 displays the temperature type selection screen 42 on the display device 4 (see FIG. 6). Then, in step S42, the input for selecting the temperature type is accepted, and in step S43, the result of the input in step S42 is determined. In step S43, if the input result is “during heating,” the temperature type is set to “during heating” in step S44 and the process returns (i.e., proceeds to step S5 in FIG. 8). If the input result is “during cooling” in step S43, the temperature type is set to “during cooling” in step S45 and the process returns. If the input result is “both” in step S43, the temperature type is set to “both” in step S44 and the process returns.

In the training data extraction processing in step S5, as shown in FIG. 12, the training data extraction processing unit 25 accepts inputs for the settings of the explanatory variables and the objective variables in step S51. If the settings of the explanatory variables and the objective variables are predetermined, step S51 can be omitted. Thereafter, the temperature type is determined in step S52. If the temperature type is “during heating,” in step S53, the training data 32 including the Curie temperature during heating T_{C_H} as the structure data 64 is extracted, and the process proceeds to step S56. When the temperature type is “during cooling,” in step S54, the training data 32 including the Curie temperature during cooling T_{C_C} as the structure data 64 is extracted, and then the process goes to step S56. If the temperature type is “both,” the training data 32 including the Curie temperature during heating T_{C_H} and the Curie temperature during cooling T_{C_C} as the structure data 64 is extracted, and proceed to step S56. In step S56, the extracted training data 32 is stored in the storage unit 3, and then the process returns and proceeds to step S6 in FIG. 8.

In the regression model creation processing of step S6, as shown in FIG. 13A, first, in step S61, the regression model creation processing unit 26 updates the regression model 33 using the unlearned training data 32 (the explanatory variable data 71 and the objective variable data 72 extracted in step S5) for machine learning. If the regression model 33 has not yet been created, step S61 is used to create a new regression model 33. Then, in step S62, the updated (or created) regression model 33 is stored in the storage unit 3, and then the process returns.

When estimating the desired data, the estimation source data 34 is input using the input device 5 or other devices (step S11). The data to be used as the estimation source data 34 may be input to the material data processing device 1 in advance, and the input device 5 may be configured to select the data to be used as the estimation source data 34.

In step S7, the control unit 2 determines whether the estimation source data 34 has been input. If NO (N) is determined in step S5, the process returns (returns to step S1). If YES (Y) is determined in step S7, the process proceeds to step S8.

In step S8, the estimation processing is performed. In the estimation processing, as shown in FIG. 13B, first, in step S81, the estimation processing unit 27 obtains the estimated data 35 corresponding to the estimation source data 34 using the regression model 33. Then, in step S82, the obtained estimated data 35 is stored in the storage unit 3. Then, the process returns and proceeds to step S9 in FIG. 8.

In step S9, the estimated data presentation processing is performed. In the estimated data presentation processing, for example, the estimated data presentation processing unit 28 presents the estimated data 35 estimated in step S8 by displaying the estimated data 35 on the display device 4. After that, the process returns (returns to step S1).

(Setting of the Explanatory Variables and the Objective Variables)

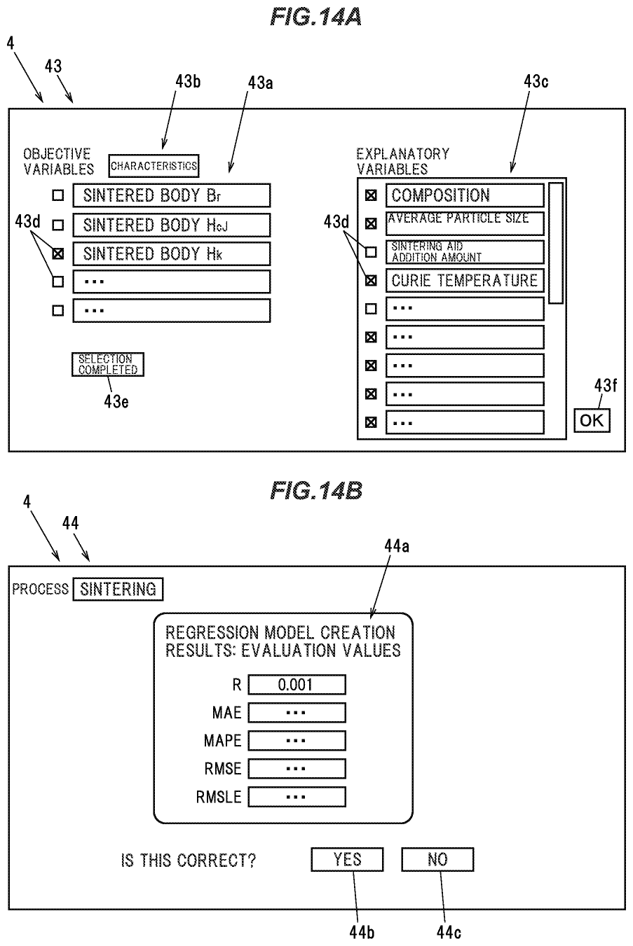

FIG. 14A is a diagram showing an example of a variable setting screen 43 that is displayed when creating the regression model 33. As shown in FIG. 14A, the variable setting screen 43 has an objective variable selection area 43a for selecting the objective variable on the left side of the screen and an explanatory variable selection area 43c for selecting the explanatory variable on the right side of the screen. The user first selects which of the process data 61, the composition data 62, the characteristics data 63, and the structure data 64 is to be used as the objective variable in the data selection area 43b of the objective variable selection area 43a. Then, the items of the objective variable corresponding to the selection are displayed in the objective variable selection area 43a. The user then selects the objective variable by checking checkboxes 43d in the objective variable selection area 43a, and presses (clicks or touches) a selection complete button 43e. Then, the appropriate explanatory variables are displayed in the explanatory variable selection area 43c with the appropriate explanatory variables already checked according to the selected objective variable. The user decides which explanatory variable to use by adding or removing a check from the checkbox 43d in the explanatory variable selection area 43c, and then presses an OK button 43f. The temperature type selection screen 42 shown in FIG. 6 is then displayed, and based on the selection results of the variable setting screen 43 and the temperature type selection screen 42, the above training data extraction processing and the regression model creation processing are performed to create the regression model 33.

Although not mentioned above, if, for example, too many explanatory variables are used, the estimation accuracy of the regression model 33 may decrease due to over-training. Therefore, in this embodiment, the control unit 2 is configured to create the regression model 33 using a portion (e.g., 70%) of the training data 32, conduct a test using the remaining portion (e.g., 30%) of the training data 32, and display the evaluation values on the display device 4. FIG. 14B is a diagram showing an example of the evaluation value display screen 44 that displays the evaluation values. As shown in FIG. 14B, the evaluation value display screen 44 has an evaluation value display area 44a in the center of its screen, where the evaluation value is displayed. Which evaluation value is used can be changed as needed. At the bottom of the evaluation value display screen 44, a message prompting confirmation, such as “Is this correct?”, is displayed. If a Yes button 44b is pressed, for example, the screen moves to the screen for inputting the source data. If a No button 44c is pressed, the screen returns to the variable setting screen 43 in FIG. 14B, where the objective variables and explanatory variables to be used are set once again. By repeating the selection and evaluation of the objective variables and explanatory variables to be used, the appropriate objective variables and explanatory variables can be selected and the estimation accuracy can be improved.

The screens shown in FIGS. 14A and 14B are only examples, and the display layout, display items, display format, etc. can be changed as necessary.

### Function and Effect of the Embodiment

As explained above, in the material data processing device 1 according to this embodiment, the structure data 64 includes a feature amount based on the magnetization temperature dependence during heating and a feature amount based on the magnetization temperature dependence during cooling, and is provided with a temperature type selection means 10 for selecting the use of either one or both of the feature amounts based on the magnetization temperature dependence during heating or the feature amounts based on the magnetization temperature dependence during cooling as the structure data 64 used for machine learning.

Conventionally, the structure data 64 has often been obtained using XRD, SEM/EDX, or EPMA. However, when using SEM/EDX or EPMA, if the size of the phase of interest in the material is extremely small, it may be difficult to obtain accurate information because the compositional information of another phase surrounding the phase of interest may be superimposed due to the spread of the incident electron beam. Furthermore, the quality of data may vary greatly depending on the skill and subjectivity of the observer (which area is evaluated) during sample preparation and observation. Furthermore, when using SEM/EDX or EPMA, complicated procedures such as image processing are required to obtain phase ratios and composition of each phase from the obtained data, making it difficult to obtain a large amount of data necessary for data science use. In addition, with the method obtained by XRD, for example, in magnetic materials, differences in the crystal structures of different phases in the same material may be reflected only in the presence or absence of specific superlattice reflections, making it difficult to detect when the phase of interest exists only in trace amounts. Also, when multiple phases with the same crystal structure but different compositions coexist in a material, it is difficult to separate them. Thus, with the conventional method, it was difficult to efficiently and sensitively acquire data on the structure of materials whose properties are sensitively affected by fine constituent phases, especially magnetic materials, without relying heavily on the skill and subjectivity of the measurer.

In contrast, the structure data 64 used in this embodiment are feature amounts based on magnetization temperature dependence. This feature amount based on magnetization temperature dependence is relatively easy to acquire data because the quality of data is unlikely to fluctuate depending on the personal skills of the person collecting the data, and the data can be acquired mechanically. By using the feature amounts based on magnetization temperature dependence as the feature amounts of “structure,” it will be possible to construct mathematical models that could not be constructed from conventional databases, and it is expected to promote the development of materials through materials informatics.

Furthermore, in this embodiment, it is selectable to use either one or both of the feature amounts based on the magnetization temperature dependence during heating or the feature amounts based on the magnetization temperature dependence during cooling as the structure data 64 used for machine learning. This makes it possible to appropriately select the feature amount based on the magnetization temperature dependence to be used as the structure data 64 according to the type of magnet, etc., thereby improving the accuracy of the estimation. In other words, according to this embodiment, it is possible to realize a material data processing device 1 that is easy to acquire data and enables highly accurate estimation.

### Summary of Embodiment

Next, the technical concepts that can be grasped from the above embodiment will be described with the help of the codes, etc. in the embodiment. However, each sign, etc. in the following description is not limited to the members, etc. specifically shown in the embodiment for the constituent elements in the scope of claims.

According to the first feature, a material data processing device 1 using a computer includes a regression model creation processing unit 26 that performs machine learning using, out of process data 61 including information on manufacturing conditions for manufacturing individual samples, composition data 62 including information on composition of the individual samples, characteristics data 63 including information on characteristics of the individual samples, and structure data 64 including information on structure of the individual samples, two or more data including the structure data 64, and creates a regression model 33 representing a correlation between respective data; an estimation processing unit 27 that estimates, by using the regression model 33, the process data 61, the composition data 62, the characteristics data 63, or the structure data 64, having been used for machine learning, wherein the structure data 64 includes a feature amount based on a magnetization temperature dependence during heating, and a feature amount based on a magnetization temperature dependence during cooling; and a temperature type selection means 10 that selects use of either one or both of the feature amount based on the magnetization temperature dependence during heating or the feature amount based on the magnetization temperature dependence during cooling as the structure data 64 to be used for the machine learning.

According to the second feature, in the material data processing device 1 as described in the first feature, the regression model creation processing unit 26 creates the regression model 33 with the characteristics data 63 as objective variable data 72 and data other than the characteristics data 63 as explanatory variable data 71.

According to the third feature, the material data processing device 1 as described in the first or second feature further includes a feature amount extraction processing unit 23 extracts, based on measured data 641 of the individual samples, the feature amount based on the magnetization temperature dependence during heating and the feature amount based on the magnetization temperature dependence during cooling, wherein the feature amount extraction processing unit 23 divides the measured data 641 into measured data during heating and measured data during cooling, and extracts the feature amount based on the magnetization temperature dependence during heating from the measured data during heating and the feature amount based on the magnetization temperature dependence during cooling from the measured data during cooling.

According to the fourth feature, in the material data processing device 1 as described in any one of the first to third features, the feature amount based on the magnetization temperature dependence is a feature amount related to magnetic phase transition.

According to the fifth feature, in the material data processing device as described in the fourth feature, the feature amount based on the magnetization temperature dependence includes at least one of Curie temperature and Neel temperature.

According to the sixth feature, in the material data processing device 1 as described in any one of the first to fifth features, the composition data 62 includes the types of elements contained in the individual samples and composition ratios of the elements, and the process data 61 includes parameters defining heat treatment conditions.

According to the seventh feature, in the material data processing device 1 as described in any one of the first to sixth features, the characteristics data includes at least one of residual flux density, coercive force, saturation magnetization, and magnetic permeability.

According to the eighth feature, in the material data processing device 1 as described in any one of the first to seventh features, the structure data 64 includes a parameter defining a crystal structure of a main phase.

According to the ninth feature, a computer-performed material data processing method includes performing machine learning using, out of process data 61 including information on manufacturing conditions for manufacturing individual samples, composition data 62 including information on composition of the individual samples, characteristics data 63 including information on characteristics of the individual samples, and structure data 64 including information on structure of the individual samples, two or more data including the structure data 64, and creating a regression model 33 representing a correlation between respective data; estimating, by using the regression model 33, the process data 61, the composition data 62, the characteristics data 63, or the structure data 64, having been used for machine learning, wherein the structure data 64 includes a feature amount based on a magnetization temperature dependence during heating, and a feature amount based on a magnetization temperature dependence during cooling; and selecting use of either one or both of the feature amount based on the magnetization temperature dependence during heating or the feature amount based on the magnetization temperature dependence during cooling as the structure data 64 to be used for the machine learning.

According to the tenth feature, a material data processing device 1 using a computer includes a regression model creation processing unit 26 that performs machine learning using, out of process data 61 including information on manufacturing conditions for manufacturing individual samples, composition data 62 including information on composition of the individual samples, characteristics data 63 including information on characteristics of the individual samples, and structure data 64 including information on structure of the individual samples, two or more data including the structure data 64, and creates a regression model 33 representing a correlation between respective data; and an estimation processing unit 27 that estimates, by using the regression model, the process data 61, the composition data 62, the characteristics data 63, or the structure data 64, having been used for machine learning, wherein the structure data 64 includes a feature amount based on a magnetization temperature dependence during heating, and a feature amount based on a magnetization temperature dependence during cooling.

The above description of the embodiments of the invention does not limit the invention as claimed above. It should also be noted that not all of the combinations of features described in the embodiments are essential to the means for solving the problems of the invention. In addition, the invention can be implemented with appropriate modifications to the extent that it does not depart from the gist of the invention.

1. A material data processing device using a computer, comprising:
a regression model creation processing unit that performs machine learning using, out of process data including information on manufacturing conditions for manufacturing individual samples, composition data including information on composition of the individual samples, characteristics data including information on characteristics of the individual samples, and microstructure data including information on microstructure of the individual samples, two or more data including the microstructure data, and creates a regression model representing a correlation between respective data;
an estimation processing unit that estimates, by using the regression model, the process data, the composition data, the characteristics data, or the microstructure data, having been used for machine learning, wherein the microstructure data includes a feature amount based on a magnetization temperature dependence during heating, and a feature amount based on a magnetization temperature dependence during cooling; and
a temperature type selection means that selects use of either one or both of the feature amount based on the magnetization temperature dependence during heating or the feature amount based on the magnetization temperature dependence during cooling as the structure data to be used for the machine learning.

2. The material data processing device according to claim 1, wherein the regression model creation processing unit creates the regression model with the characteristics data as objective variable data and data other than the characteristics data as explanatory variable data.

3. The material data processing device according to claim 1, further comprising:
a feature amount extraction processing unit extracts, based on measured data of the individual samples, the feature amount based on the magnetization temperature dependence during heating and the feature amount based on the magnetization temperature dependence during cooling,
wherein the feature amount extraction processing unit divides the measured data into measured data during heating and measured data during cooling, and extracts the feature amount based on the magnetization temperature dependence during heating from the measured data during heating and the feature amount based on the magnetization temperature dependence during cooling from the measured data during cooling.

4. The material data processing device according to claim 1, wherein the feature amount based on the magnetization temperature dependence is a feature amount related to magnetic phase transition.

5. The material data processing device according to claim 1, wherein the feature amount based on the magnetization temperature dependence includes at least one of Curie temperature and Neel temperature.

6. The material data processing device according to claim 1, wherein the composition data includes the types of elements contained in the individual samples and composition ratios of the elements, and the process data includes parameters defining heat treatment conditions.

7. The material data processing device according to claim 1, wherein the characteristics data includes at least one of residual flux density, coercive force, saturation magnetization, and magnetic permeability.

8. The material data processing device according to claim 1, wherein the microstructure data includes a parameter defining a crystal structure of a main phase.

9. A computer-performed material data processing method, comprising:
performing machine learning using, out of process data including information on manufacturing conditions for manufacturing individual samples, composition data including information on composition of the individual samples, characteristics data including information on characteristics of the individual samples, and microstructure data including information on microstructure of the individual samples, two or more data including the microstructure data, and creating a regression model representing a correlation between respective data;
estimating, by using the regression model, the process data, the composition data, the characteristics data, or the microstructure data, having been used for machine learning, wherein the microstructure data includes a feature amount based on a magnetization temperature dependence during heating, and a feature amount based on a magnetization temperature dependence during cooling; and selecting use of either one or both of the feature amount based on the magnetization temperature dependence during heating or the feature amount based on the magnetization temperature dependence during cooling as the microstructure data to be used for the machine learning.

10. A material data processing device using a computer, comprising:
a regression model creation processing unit that performs machine learning using, out of process data including information on manufacturing conditions for manufacturing individual samples, composition data including information on composition of the individual samples, characteristics data including information on characteristics of the individual samples, and microstructure data including information on microstructure of the individual samples, two or more data including the microstructure data, and creates a regression model representing a correlation between respective data; and
an estimation processing unit that estimates, by using the regression model, the process data, the composition data, the characteristics data, or the microstructure data, having been used for machine learning, wherein the microstructure data includes a feature amount based on a magnetization temperature dependence during heating, and a feature amount based on a magnetization temperature dependence during cooling.
